(19) United States
(12) Patent Application Publication
(10) Pub. No.: US 2024/0156723 A1
(43) Pub. Date: May 16, 2024

(54) DEPOT COMPOSITIONS FOR VESICULAR MONOAMINE TRANSPORTER 2 (VMAT2) INHBITORS

(71) Applicant: Foresee Pharmaceuticals Co., Ltd., Taipei City (TW)

(72) Inventors: Yuhua LI, Newark, DE (US); MingHsin Li, Taipei City (TW)

(21) Appl. No.: 18/510,915

(22) Filed: Nov. 16, 2023

Related U.S. Application Data
(60) Provisional application No. 63/384,008, filed on Nov. 16, 2022.

(51) Int. Cl. A61K 9/00; A61K 9/06; A61K 31/4745; A61K 47/24; A61K 47/28

(52) U.S. Cl. CPC A61K 9/0024; A61K 9/06; A61K 31/4745; A61K 47/24; A61K 47/28

(57) ABSTRACT

The present invention provides a depot composition suitable to deliver a VMAT2 inhibitor in a controlled manner. The composition of the present invention comprises: (a) a VMAT2 inhibitor and (b) a pharmaceutically acceptable oil. Optionally, the depot composition of the present invention also comprises an excipient to achieve optimal delivery of the VMAT2 inhibitor. The present invention also provides a method for making such depot composition.

### CROSS REFERENCE TO RELATED APPLICATION

This application is entitled to priority to U.S. Provisional Patent Application No. 63/384,008, filed Nov. 16, 2022, the disclosure of which is incorporated herein by reference.

### FIELD OF THE INVENTION

The field of the invention relates to a depot composition for the sustained release delivery of vesicular monoamine transporter 2 (VMAT2) inhibitors. The depot composition comprises a VMAT2 inhibitor, a lipid, and a pharmaceutically acceptable oil. The invention also relates to a process for making such a depot composition and a method of use thereof.

### BACKGROUND OF THE INVENTION

Vesicular monoamine Type 2 (VMAT2) receptor is a membrane protein that transports monoamine neurotransmitters, such as dopamine, serotonin and histamine, from presynaptic into synaptic vesicles. By blocking the VMAT2 receptor, the level of monoamine in the central neural system could be down-regulated. This down-regulation is believed to offer a mechanism to treat a variety of hyperkinetic movement disorders. Various VMAT2 inhibitors have been studied as therapeutic agents to treat different hyperkinetic movement disorders, such as Tardive Dyskinesia (TD), Tourette syndrome, and Huntington's disease [Sulzer D., et al., Prog. Neurobiol., 2005, April, 75 (6): 406-33].

Tetrabenazine (TBZ) or 1,3,4,6,7,11b-hexahydro-9,10-dimethoxy-3-(2-methoxylrpopryl)-2H-benzo(a)quinoline-2-one) is a potent and reversible VMAT2 inhibitor with a binding affinity of Ki-100 nM. The compound has been known since the 1950s. TBZ was approved for the symptomatic treatment of chorea associated with Huntington's disease on Aug. 15, 2008. It is sold under the brand names Nitoman and Xenazine® among others. Although other drugs had been used “off label”, tetrabenazine was the first approved treatment for Huntington's disease in the U.S. [Frank S., Neuropsychiatr. Ds. Treat 2010, 6:657-65].

Xenazine® contains a racemic mixture of (+)-TBZ and (−)-TBZ. The racemic TBZ is rapidly metabolized (majorly in the liver by carbonyl reductase) into four stereoisomeric dihydrotetrabenazine (DHTBZ) metabolites: (+)-(α)-DHTBZ, (+)-(β)-DHTBZ, (−)-(α)-DHTBZ, and (−)-(β)-DHTBZ (Skor H., et al., Drugs R. D., 2017 September, 17(3):449-459). The human VMAT2 binding affinity Ki of these four metabolites is about 4.2, 9.7, 250, and 690 nM, respectively [Grigoriadis, et al., Journal of Pharmacology and Experimental Therapeutics June 2017, 361 (3): 454-461]. In addition, (−)-(α)-DHTBZ, and (−)-(β)-DHTBZ have high off-target binding affinity to dopamine D2 and serotonin 5-HT7 receptors (180/71 nM and 53/5.9 nM for ((−)-α) and ((−)-β), respectively), which results in severe side effects of TBZ administration (i.e. insomnia, tremor, rigid muscle, problems with balance etc.) [Harriott, et al., Progress in Medicinal Chemistry Volume 57, 2018, Pages 87-111]. Moreover, due to the variable CYP 2D6-mediated metabolism of TBZ, the maintenance dose of TBZ varies from one individual to another, therefore, CYP 2D6 inducers or inhibitors should also be avoided for subjects taking TBZ. Furthermore, the low oral bioavailability (17%) and short circulation half-life led to the consequence of a large dose level and multiple doses daily. Therefore, there is a need to develop alternative VMAT2 inhibitor drugs to eliminate or minimize the drawbacks related to Xenazine®.

In 2017, two new medications were approved to treat TD: Valbenazine (VBZ) (INGREZZA®, Neurocrine Biosciences, Inc., single 40 mg, 60 mg or 80 mg capsule per day) and deutetrabenazine (d6-TBZ) (AUSTEDO®, Teva, 6 mg, 9 mg, or 12 mg tablet, twice daily). Unlike TBZ, d6-TBZ and VBZ have pharmacokinetic advantages which enable less frequent dosing for better tolerability. VBZ, L-Valine, (2R, 3R, 11bR)-1, 3, 4, 6, 7, 11b-hexahydro-9,10-dimethoxy-3-(2-methylpropyl)-2H-benzo[α]quinolizin-2-yl ester, is an ester of (+)-(α)-DHTBZ with the amino acid L-valine. VBZ facilitated by conjugating with valine, increases the bioavailability to ˜50%, compared to ˜17% for TBZ. By solely introducing (+)-(α)-DHTBZ without the presence of the other side effect inducing stereoisomeric metabolites, such as (−)-(α)-DHTBZ and (−)-(β)-DHTBZ, VBZ is considered much more tolerable and safer than TBZ. On the other hand, in the case of AUSTEDO®, the deuterated derivative of TBZ increases the half-life of d6-TBZ which benefits for the reduced dosing frequency.

Although TBZ, d6-TBZ and VBZ were approved for treating hyperkinetic diseases including chorea associated with Hungtinton's disease and TD, the frequent dosing for chronic medication causes significant challenge of patient compliance. Per regimen recommended by label, oral TBZ, d6-TBZ and VBZ are prescribed three times, twice and once daily, respectively. The potential non-adherence is further aggravated when the patient needs to take multiple medicines, such as antipsychotic drugs. There is still a medical need for a drug which significantly improves the patient's safety and compliance, with improved efficacy.

### SUMMARY OF THE INVENTION

The present invention provides a depot composition suitable for the sustained release delivery of VMAT2 inhibitors. The invented composition is particularly featured with a high content of VMAT2 inhibitors, which enables a sufficient drug supply in a compact volume. Another inventive feature is the non-polymeric controlled-release depot composition, comprising pharmaceutically acceptable and chemically well-characterized inactive ingredients. The depot composition behaves as a shear-thinning material, which is particularly favorable, leading to a stable uniform suspension and low injection force. The depot composition of the present invention is pre-filled in a ready-to-use syringe, without the need of solid-liquid reconstitution or mixing. The invention also provides a process for making such a depot composition and a method of use thereof.

In one general aspect, the present invention provides a depot composition for controlled release delivery comprising: a) a vesicular monoamine transporter 2 (VMAT2) inhibitor or a pharmaceutically acceptable salt thereof; b) a pharmaceutically acceptable oil; and c) optionally a lipid. Preferably, the lipid is a solid at a temperature between about 15 and about 30° C.

According to the present invention, the depot composition is produced by a process comprising: combining the pharmaceutically acceptable oil and the VMAT2 inhibitor or the pharmaceutically acceptable salt thereof, and optionally combining with the lipid, to form a depot composition. The depot composition can be prepared by mixing using conventional methods. The depot composition may be filled into a syringe to facilitate administration and may be preferably prefilled into a single syringe in a ready-to-use configuration.

In another aspect, the present invention provides a method of treating a hyperkinetic movement disorder comprising administering a patient in need thereof the depot composition described herein.

The details of one or more embodiments of the invention are set forth in the description below. Other features and advantages will be apparent from the following detailed description, and the appended claims.

### BRIEF DESCRIPTION OF THE DRAWINGS

The foregoing and other objects, aspects, features, and advantages of exemplary embodiments will become more apparent and may be better understood by referring to the following description taken in conjunction with the accompanying drawings.

FIG. 1. Microscopy of recrystallized (+)-TBZ (a) and racemic (±)-d6-TBZ (b) in 3-gram batches.

FIG. 2. Microscopy of recrystallized (+)-TBZ in the 12-gram batch.

FIG. 3. Microscopy of spray dried cholesterol.

FIG. 4. Microscopy of cholesterol fibers in oleogel.

FIG. 5. Microscopy of VMAT2 inhibitor formulation, F-08, and morphology of (+)-TBZ crystals.

FIG. 6. Viscosity-shear rate profile of VMAT2 inhibitor formulation, F-08.

FIG. 7. Dissolution profiles of VMAT2 inhibitor formulations, F-06 (50 wt % rex (+)-TBZ/15 wt % SPD CSTR/35 wt % SSO), F-07 (50 wt % rex (+)-TBZ/10 wt % CSTR fiber/40 wt % SSO), F-07b (50 wt % rex (+)-TBZ/15 wt % CSTR fiber/35 wt % SSO) and F-08 (50 wt % (+)-TBZ/10% CSTR fiber/40% wt SSO).

FIG. 8. 2-week of PK profiles of VMAT2 inhibitor formulations, F-01 (50 wt % rex (+)-TBZ/15 wt % raw CSTR/35 wt % SSO) and F-07b (50 wt % rex (+)-TBZ/15 wt % CSTR fiber/35 wt % SSO). Dn: dose-normalized.

FIG. 9. 5-week of PK profiles of VMAT2 inhibitor formulations, F-06 (50 wt % rex (+)-TBZ/15 wt % SPD CSTR/35 wt % SSO), F-07 (50 wt % rex (+)-TBZ/10 wt % CSTR fiber/40 wt % SSO), F-07b (50 wt % rex (+)-TBZ/15 wt % CSTR fiber/35 wt % SSO) and F-07d (50 wt % rex d6-TBZ/15 wt % CSTR fiber/35 wt % SSO). Dn: dose-normalized.

FIG. 10. PK profiles of VMAT2 inhibitor formulations, F-08 with three dose levels. L: low. M: middle. H: high. Composition of F-08: 50 wt % (+)-TBZ/10% CSTR fiber/40% wt SSO.

FIG. 11. PK profiles of VMAT2 inhibitor formulations, F-06 (50 wt % rex (+)-TBZ/15 wt % SPD CSTR/35 wt % SSO) in dogs. Dn: dose-normalized.

FIG. 12. PK profiles of F-DH-01 in male rats. Dose=60 mg/kg

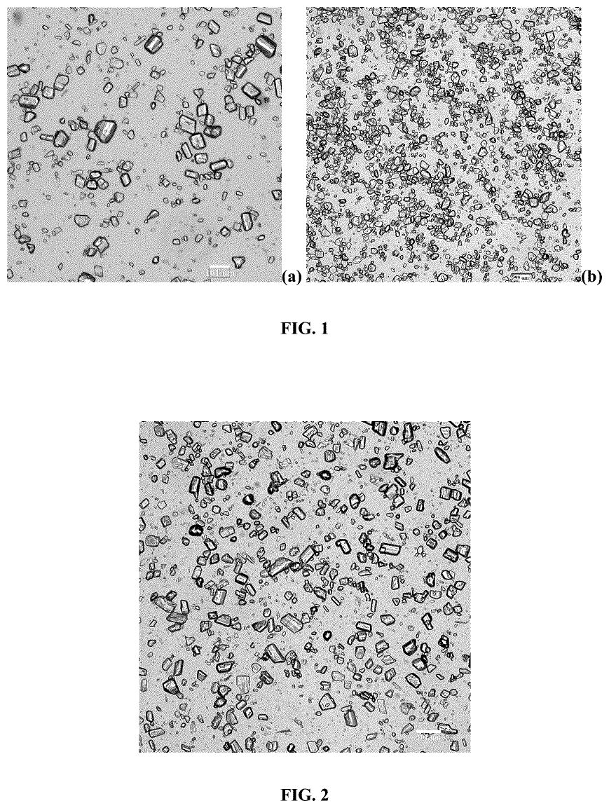

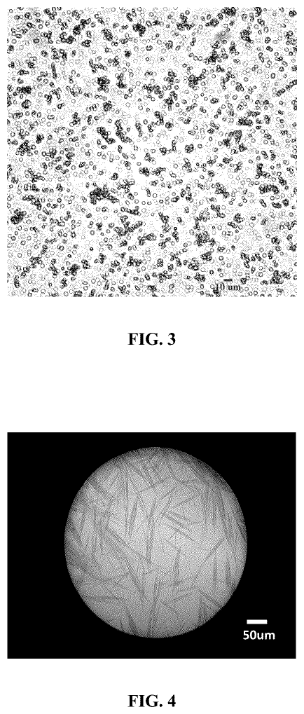

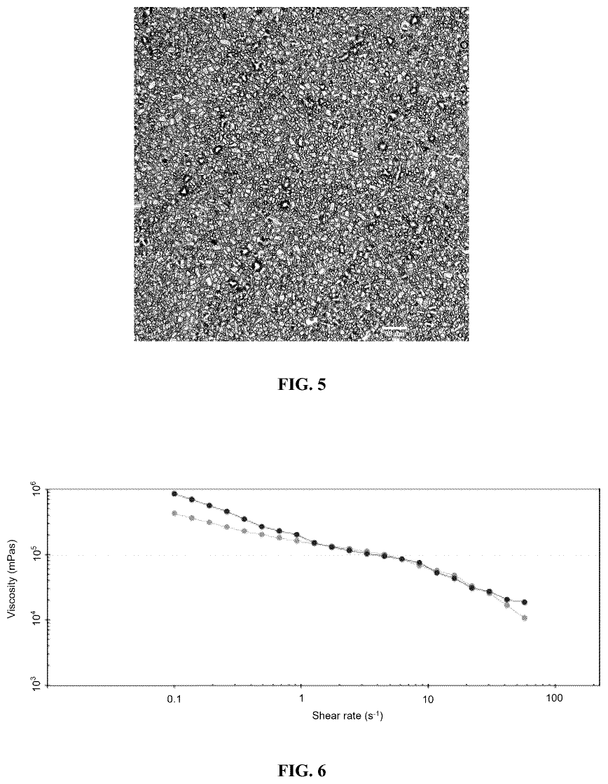

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

### Definitions

As used herein, the singular forms “a,” “an,” and “the” and similar referents are to be construed to cover both the singular and the plural, unless otherwise indicated herein or clearly contradicted by context.

As used herein, in the context of the present invention, all numbers disclosed herein are approximations, whether or not the words “about” or “approximately” are used. Each numerical number means a range of the numerical value ±10% of the numerical value unless otherwise indicated. For example, “about 100 mL” or “100 mL” includes any values between 90 and 110 mL.

As used herein, the term “about” or “approximately” preceding a numerical value or a series of numerical values means±10% of the numerical value unless otherwise indicated. For example, “approximately 100 mg” means 90 to 110 mg.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the invention can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, “treatment” or “treat” refers to the treatment of a disease, disorder, or medical condition (such as a gastrointestinal inflammatory disease), in a patient, such as a mammal (particularly a human) which includes one or more of the following:
(a) preventing the disease, disorder, or medical condition from occurring, i.e., preventing the reoccurrence of the disease or medical condition or prophylactic treatment of a patient that is pre-disposed to the disease or medical condition;
(b) ameliorating the disease, disorder, or medical condition, i.e., eliminating or causing regression of the disease, disorder, or medical condition in a patient, including counteracting the effects of other therapeutic agents;
(c) suppressing the disease, disorder, or medical condition, i.e., slowing or arresting the development of the disease, disorder, or medical condition in a patient; or
(d) alleviating the symptoms of the disease, disorder, or medical condition in a patient.

As used herein, the term depot composition refers to a slow-release, or sustained release, or controlled release formulation of an active ingredient which releases the active ingredient over a long period of time to permit less frequent administration. The depot compositions are designed to increase medication adherence and consistency, especially in patients who may forget to take their medicine.

As used herein, the vesicular monoamine transporter type 2 (VMAT2) inhibitors are defined as the agents that cause a depletion of neuroactive peptides such as dopamine in nerve terminals and are used to treat chorea due to neurodegenerative diseases (such as Huntington chorea) or dyskinesias due to neuroleptic medications (tardive dyskinesia). Some examples are described in US patents U.S. Pat. Nos. 9,714,246, 9,988,382, and 11,053,242.

As used herein, the term of TBZ is defined as tetrabenazine, (±)-TBZ or 1,3,4,6,7,11b-hexahydro-9,10-dimethoxy-3-(2-methoxylrpopryl)-2H-benzo(a)quinoline-2-one).

As used herein, the term of (+)-TBZ is defined as (+)-tetrabenazine, (3R,11bR)-TBZ, or (3R,11bR)-tetrabenazine. It is a reversible inhibitor of vesicular monoamine transporter 2 (VMAT-2).

As used herein, the term of (−)-TBZ is defined as (−)-tetrabenazine, (3R,11bS)-TBZ, or (3R,11bS)-tetrabenazine.

As used herein, the term of VBZ is defined as valbenazine or L-Valine, (2R,3R,11bR)-1,3,4,6,7,11b-hexahydro-9,10-dimethoxy-3-(2-methylpropyI)-2H-benzo[a]quinolizin-2-yl ester.

As used herein, the term of (±)-d6-TBZ is defined as deutetrabenazine, or racemic deutetrabenazine. Deutetrabenazine is a hexahydro-dimethoxybenzoquinolizine derivative and has the following chemical name: (RR, SS)-1,3,4,6,7,11b-hexahydro-9, 10-di(methoxy-d3)-3-(2-methylpropyl) 2H-benzo[a]quinolizin-2-one. Deutetrabenazine is a racemic mixture containing RR-deutetrabenazine ((+)-d6-TBZ) and SS-deutetrabenazine ((−)-d6-TBZ).

As used herein, the term of (+)-d6-TBZ is defined as RR-deutetrabenazine and the term of (−)-d6-TBZ is defined as SS-deutetrabenazine.

As used herein, the term of (+)-(α)-DHTBZ is defined as [+]-α-dihydrotetrabenazine, one of the metabolites of tetrabenazine.

As used herein, the term of (+)-(β)-DHTBZ is defined as H-β-dihydrotetrabenazine, one of the metabolites of tetrabenazine.

As used herein, the term of (−)-(α)-DHTBZ is defined as [−]-α-dihydrotetrabenazine, one of the metabolites of tetrabenazine.

As used herein, the term of (−)-(β)-DHTBZ is defined as [−]-β-dihydrotetrabenazine, one of the metabolites of tetrabenazine.

As used herein, the term of (+)-d6-(α)-DHTBZ is defined as (+)-d6-alpha-dihydrotetrabenazine, one of the metabolites of deutetrabenazine.

As used herein, the term of (−)-d6-(α)-DHTBZ is defined as (−)-d6-alpha-dihydrotetrabenazine, one of the metabolites of deutetrabenazine.

As used herein, the term of (+)-d6-(β)-DHTBZ is defined as (+)-d6-beta-dihydrotetrabenazine, one of the metabolites of deutetrabenazine.

As used herein, the term of (−)-d6-(β)-DHTBZ is defined as (−)-d6-beta-dihydrotetrabenazine, one of the metabolites of deutetrabenazine.

As used herein, the term of SSO is defined as sesame oil.

As used herein, the term of CTO is defined as castor oil.

As used herein, the term of MCT is defined as medium-chain triglycerides.

As used herein, the term of SPC is defined as soybean phosphatidylcholine.

As used herein, the term of CSTR is defined as cholesterol.

When the VMAT2 inhibitor or the pharmaceutically acceptable salt thereof is a powder, the particle size distribution is an important quality parameter, because many other product properties are directly related to it. Particle size distribution influences material properties like flowability and conveying behavior (for bulk materials), reactivity, abrasiveness, solubility, extraction and reaction behavior, taste, compressibility, and many more.

Depending on the material characteristics and the scope of the examination, various conventional methods can be used to obtain particle size distribution. These include Laser Diffraction (LD), Dynamic Light Scattering (DLS), Dynamic Image Analysis (DIA) or Sieve Analysis. A used herein, Malvern Mastersizer 2000 or 3000 laser particle size analyzer or equivalent instrument is preferably used to analyze the particle size distribution of the powders.

As used herein, the particle size distribution of powders is typically expressed by D10, D50, and D90 where “D” means Distribution. D10 represents that 10% of particles in the powders are smaller than this size. Typically, the unit is micrometer (μm). It can also be represented by a statistical symbol such as Dv10 or Dv0.1. V means volume based. Dv90 or Dv(0.9) means that 90% of the total particles are smaller than this size based on volume. Dv50 or Dv(0.5) means that 50% of the total particles are smaller than this size. Dv50 is the median particle size distribution. As used herein, the term “D[4,3]” refers to the volume mean or average diameter of the particles.

As used herein, the term of pharmaceutically acceptable oil is intended to includes any triglyceride oil, canola oil, castor oil, corn oil, cottonseed oil, grapeseed oil, olive oil, palm oil, peanut oil, rapeseed oil, safflower oil, sesame oil, soybean oil, sunflower oil, “vegetable” oil, walnut oil, medium-chain triglyceride (MCT) oil, and the like.

As used herein, the term of lipid in a broader sense, is intended to include triglycerides (e.g., tristearin), diglycerides (e.g., glycerol bahenate), monoglycerides (e.g., glycerol monostearate), fatty acids (e.g., stearic acid), steroids (e.g., cholesterol), and waxes (e.g., cetyl palmitate). Preferably, the lipid is a solid at a temperature between 15 to 30° C.

As used herein, the term of pharmaceutically acceptable salt means a salt that is acceptable for administration to a human subject, e.g., salts having acceptable mammalian safety for a given dosage regime. Representative pharmaceutically acceptable salts include salts of acetic, ascorbic, benzenesulfonic, benzoic, camphorsulfonic, citric, ethanesulfonic, edisylic, fumaric, gentisic, gluconic, glucoronic, glutamic, hippuric, hydrobromic, hydrochloric, isethionic, lactic, lactobionic, maleic, malic, mandelic, methanesulfonic, mucic, naphthalenesulfonic, naphthalene-1,5-disulfonic, naphthalene-2,6-disulfonic, nicotinic, nitric, orotic, pamoic, pantothenic, phosphoric, succinic, sulfuric, tartaric, p-toluenesulfonic and xinafoic acid, and the like.

As used herein, the term “controlled release delivery” is intended to refer to the delivery of a VMAT2 inhibitor or a pharmaceutically acceptable salt thereof in vivo over a desired, extended period of time following administration, preferably from at least a few days, a few weeks, or a few months to one year.

### Depot Compositions

In a general aspect, the present invention relates to a depot composition suitable for the sustained release delivery of VMAT2 inhibitors. In one embodiment, the depot composition for controlled release delivery of vesicular monoamine transporter 2 (VMAT2) inhibitors comprises: a) a VMAT2 inhibitor or a pharmaceutically salt thereof; b) a pharmaceutically acceptable oil; and c) optionally a lipid.

In some embodiments, the depot composition comprises the lipid. Preferably, the lipid is a solid at a temperature between about 15 and about 30° C.

In some embodiments, the depot composition does not comprise the lipid.

In certain embodiments, the VMAT2 inhibitor in the depot composition of the present invention is selected from a group consisting of VBZ, (±)-TBZ, (+)-TBZ, (−)-TBZ, (±)-d6-TBZ, (+)-d6-TBZ and (−)-d6-TBZ.

In certain embodiments, the VMAT2 inhibitor in the depot composition of the present invention is selected from a group consisting of (+)-(α)-DHTBZ, (−)-(α)-DHTBZ, (+)-(β)-DHTBZ, (−)-(β)-DHTBZ, (+)-d6-(α)-DHTBZ, (−)-d6-(α)-DHTBZ, (+)-d6-(β)-DHTBZ, and (−)-d6-(β)-DHTBZ.

In certain embodiments, the VMAT2 inhibitor in the depot composition of the present invention is a combination of any two or more VMAT2 inhibitors as defined in the present invention.

In one preferred embodiment, the VMAT2 inhibitor in the depot composition of the present invention is (+)-TBZ.

In another preferred embodiment, the VMAT2 inhibitor in the depot composition of the present invention is (+)-d6-TBZ.

In certain embodiments, the pharmaceutically acceptable oil in the depot composition of the present invention is selected from the group consisting of sesame oil, soybean oil, cottonseed oil, castor oil, olive oil, medium chain triglyceride (MCT), dioleate glycerol, ethyl oleate and ethyl linoleate.

In one preferred embodiment, the pharmaceutically acceptable oil in the depot composition of the present invention is sesame oil or soybean oil.

In certain embodiments, the lipid in the depot composition of the present invention is selected from the group consisting of sterols, natural waxes, synthetic waxes, hydroxysteroid, and ketosteroid.

Examples of the sterols include, but not limited to, cholesterol, phytosterol, and other sterols containing a 4-ring system such as campesterol, ergosterol, sitosterol, and stigmasterol.

In one preferred embodiment, the lipid in the depot composition of the present invention is cholesterol.

In certain embodiments, the content of the VMAT2 inhibitor or the pharmaceutically acceptable salt thereof in the depot composition of the present invention is less than about 70% by weight, relative to the weight of the depot composition.

In certain embodiments, the ratio of the lipid to the pharmaceutically acceptable oil in the depot composition of the present invention is in the range from about 1:1 to 0:100, preferably from about 1:1 to 1:100, more preferably, from about 1:1 to about 1:49, by weight, relative to the weight of the depot composition. When the ratio is 0:100, it refers to a depot composition that does not comprise a lipid. In certain embodiments, the lipid in the depot composition of the present invention remains as solid particles in different forms including crystals, microspheres, microparticles and fiber-like structures. The solid lipid particles are characterized with square, rectangular, trapezoidal, irregularly shaped or rod-like appearance.

In certain embodiments, the content of the solid lipid in the depot composition of the present invention is less than about 50% or from 0% to 50% by weight, relative to the weight of the depot composition.

In certain embodiments, the solid lipid microspheres in the depot composition of the present invention are characterized with round or oval appearance.

In certain embodiments, the solid lipid fibers in the depot composition of the present invention are characterized with high aspect ratio, which is prevalently higher than 20:1. An aspect ratio as defined herein is a proportional relationship between an image's width and height. Essentially, it describes an image's shape. Aspect ratios are written as a formula of width to height, like this: 3:2. For example, a square image has an aspect ratio of 1:1, since the height and width are the same.

In certain embodiments, the solid lipid fibers are dispersed in a pharmaceutically acceptable oil to form a gel, a suspension, a paste, a semisolid, or a cream.

In certain embodiments, the VMAT2 inhibitor or the pharmaceutically acceptable salt thereof in the depot composition of the present invention is primarily presented as crystals suspended in the gel. The small amount of VMAT2 inhibitor may be dissolved in the gel. The weight ratio of VMAT2 inhibitor in solid particle forms to that dissolved in the gel is >4, preferably >10 and most preferably >20.

In certain embodiments, the ratio of the solid lipid to the pharmaceutically acceptable oil is about 1:20 to about 1:2 by weight. The viscosity is proportional to the content of solid lipid, ranging from about 1,000 to about 10,000,000 mPas, and preferably about 10,000 to about 1,000,000 mPas, as measured with a shear rate at <0.1 s⁻¹. The depot composition of the present invention is a non-flowable and shear thinning material. For example, applying a shear at <0.1 s⁻¹ to the depot composition, the viscosity is about 300,000 to about 10,000,000 mPas at room temperature. While applying a shear at >10 s⁻¹ to the same depot composition, the viscosity becomes about 1,000 to about 50,000 mPas at room temperature.

Shear thinning is a characteristic of some non-Newtonian fluids in which the fluid viscosity decreases with increasing shear stress. Shear thickening is the opposite phenomenon. By contrast to both, viscosity in Newtonian fluids is by definition independent of the forces exerted on the fluid. Fluids that exhibit shear thinning are sometimes called pseudoplastics and are typically complex fluids such as blood, motor oil, ketchup, and even whipped cream, though simple fluids can also exhibit the behavior near their critical point (NASA Science: The physics of whipped cream).

In certain embodiments, the particles of the VMAT2 inhibitor or the pharmaceutically acceptable salt thereof in the depot composition of the present invention have a particle size distribution of D90 of about 10-1,000, D50 of about 3-200, and D10 of about 0.1-50 μm, determined using a laser diffraction particle size analyzer. The particles are preferably to have D90=10-200, D50=3-100, and D10=1-25 μm. Suitable particle size and distribution of the VMAT2 inhibitor or the pharmaceutically acceptable salt thereof may be generated by dry and wet milling, such as ball mill, pin mill, hammer mill, cutting mill, mixer mill, jet mill, mortar grinders, rotor mill, disc mill, drum mill, crushers, high shear homogenization, colloid milling, precipitation, solvent evaporation, spray drying, crystallization, re-crystallization, and other conventional particle generation approaches.

In certain embodiments, the solid lipid particles in the depot composition of the present invention have a particle size distribution of D90 of about 5-1,000, D50 of about 1-100, and D10 of about 0.1-20 determined using a laser diffraction particle size analyzer. The solid lipid particles are preferably to have a D90 of about 5-200, D50 of about 2-50, and D10 of about 1-10 Suitable particle size and distribution of the VMAT2 inhibitors may be generated by dry and wet milling, such as ball mill, pin mill, hammer mill, cutting mill, mixer mill, jet mill, mortar grinders, rotor mill, disc mill, drum mill, crushers, high shear homogenization, colloid milling, precipitation, solvent evaporation, spray drying, crystallization, re-crystallization, and other conventional particle generation approaches.

According to some embodiments of the present invention, solid lipid particles are dispersed in the depot composition by high shear dispersion, rotor stator homogenization, planetary mixing or any other conventional mixing methods.

In certain embodiments, the depot composition of the present invention is sterilized using conventional methods. These methods include steam sterilization, radiation sterilization, dry heat sterilization, sterilization by filtration, gas sterilization (such as ethylene oxide or nitrogen dioxide gas sterilization sterilization), vapor sterilization, and liquid sterilization.

In certain embodiments, the degradation of the VMAT2 inhibitor or the pharmaceutically acceptable salt thereof as determined by HPLC caused by a selected sterilization method is less than about 3%, preferably less than about 1.5%, more preferably less than about 0.5%, and most preferably less than about 0.3%.

In certain embodiments, the depot composition of the present invention is stable at different temperatures. In one embodiment, impurity generation as determined by HPLC at 60° C. is less than 5% over a time period of >1 month, preferably >2 months and most preferably >3 months. In another embodiment, the impurity generation as determined by HPLC at 40° C. is less than 5% over a time period of >3 months, preferably >6 months and most preferably >9 months. In a further embodiment, the impurity generation as determined by HPLC at 25° C. is less than 5% over a time period of >18 months, preferably >24 months and most preferably >36 months.

In certain embodiments, the depot composition of the present invention delivers the VMAT2 inhibitor or the pharmaceutically acceptable salt thereof in vitro over a period of >1 week, preferably >2 weeks and most preferably >3 weeks or >4 weeks. The in vitro dissolution is conducted at 37±2° C. in 400±10 mL of PBS at pH 7.4 containing 0.2% Tween 20, with 100±10 rpm orbital agitation.

In certain embodiments, the depot composition of the present invention delivers the VMAT2 inhibitor or the pharmaceutically acceptable salt thereof in vivo over a period of >1 weeks, and preferably >4 weeks, and most preferably >8 weeks. The depot composition has a peak/-trough (P/T) ratio (Cmax/Cmin)<about 20, preferably <about 10 and most preferably <about 5.

In certain embodiments, the depot composition of the present invention can be filled into a syringe and can be easily injected via a needle of size from 18 to 23 gauges. The injection force is less than 50 N, preferably less than 30 N, and most preferably less than 20 N when a needle of 18 gauge is used.

In certain embodiments, the syringes are made of plastics including polypropylene, polycarbonate, polyethylene, cyclo-olefin-polymer (COP), and cyclic olefin copolymer (COC). The sealing stopper is made of halogenated rubber, including choloratate butyl, bromobutyl rubber. The sealing stopper can be any conventional rubber stoppers suitable for the selected syringes.

According to embodiments of the present invention, the depot composition is produced by a process comprising: combining the pharmaceutically acceptable oil and the VMAT2 inhibitor or the pharmaceutically acceptable salt thereof, and optionally combining with the lipid, to form a depot composition.

All formulations of the depot composition disclosed herein can be produced by the conventional methods in the pharmaceutical field. For example, the depot compositions can be prepared according to the methods described in the examples below.

### Methods of Use

In another general aspect, the present invention relates to a method of treating a hyperkinetic movement disorder comprising administering a patient in need thereof the depot composition described herein.

In certain embodiments, the depot composition of the present invention delivers the VMAT2 inhibitor or the pharmaceutically acceptable salt thereof over a period of >1 week, and preferably >4 weeks, and most preferably >8 weeks.

In certain embodiments, the method achieves a peak/trough (P/T) ratio <about 20, preferably <about 10, and most preferably <about 5.

In certain embodiments, the release of the VMAT2 inhibitor or the pharmaceutically acceptable salt thereof from the in situ sustained release depot is no more than about 30% of total amount of the VMAT2 inhibitor or the pharmaceutically acceptable salt thereof within 24 hours after the administration.

In certain embodiments, the hyperkinetic movement disorder is selected from the group consisting of tardive dyskinesia (TD), chorea associated with Huntington's disease (HD), tremors, dystonia, tics, myoclonus, stereotypies, restless legs syndrome, and various other disorders with abnormal involuntary movements.

### EXAMPLES

The following examples illustrate the compositions of the present invention. The examples do not limit the invention, but are provided to teach how to make useful controlled release drug delivery compositions.

### Example 1. VMAT2 Inhibitor Formulations Comprising Coarse Lipid Crystal Structured Oleogels

Preparation and Characterization of 3-Gram Batches of Recrystallized VMAT2 Inhibitors

Dissolved 3 grams of (+)-TBZ (Wuxi Pharma, China) or racemic (±)-deutetrabenazine ((±)-d6-TBZ) (Wuxi Pharma, China) in 20 mL ethanol at about 63° C. with moderate mixing in a 120-mL glass vial. Once the solution turned clear and solid particulate was absent, the temperature of the solution was cooled at a speed of ˜2° C./min by coupling a glass double-layer jacket and a water-circulation chiller. The solution was kept agitating at 200 rpm. White precipitates began to form at about 42˜45° C. and the suspension was maintained at this temperature for 1 hr. After this isothermal interval, the temperature of VMAT2 inhibitor suspension was decreased at a gradient of 10° C./50 min to 32˜35° C., followed by a temperature gradient of 4° C./10 min to 5° C. Before harvesting, the VMAT2 inhibitor suspension was equilibrated at 5° C. for 2 hours, followed by filtration with grade 5A 55 mm paper filter (Advantec, Japan) and drying at room temperature. The yield was ˜80%.

The recrystallized (+)-TBZ or (±)-d6-TBZ in 3-gram batches were observed with microscopy. Briefly, 10 mg/mL (+)-TBZ or (±)-d6-TBZ recrystallized crystal was re-dispersed in 0.2% Tween 20 deionized water with a rigorous vortex. The microscopy was conducted using the optical microscope Leica DMi8, with 100-fold magnification. The PSD was measured using laser diffraction method, with Mastersizer 3000 (Malvern, UK). Before particle sizing, the 10 mg/mL (+)-TBZ crystal was re-dispersed in 0.2% Tween 20 deionized water and sonicated for ˜10 sec.

TABLE 1
Particle size distribution (PSD) of (+)-TBZ and
racemic (±)-d6-TBZ recrystallized crystals

| VMAT2 Inhibitor | D₁₀ (μm) | D₅₀ (μm) | D₉₀ (μm) |
|---|---|---|---|
| 3 g rex (+)-TBZ-1 i | 17.3 | 38.5 | 75.0 |
| 3 g rex (+)-TBZ-1 ii | 22.0 | 45.9 | 85.7 |
| 3 g rex (+)-TBZ-1 iii | 16.6 | 33.7 | 60.7 |
| 3 g rex (±)-d6-TBZ | 12.3 | 25.6 | 45.4 |

Note:
Rex: Recrystallized; i, ii, and iii: represented three independent preparations

Formulation Composition and Compounding

One gram of various formulations were prepared in 20 mL glass vials. Compositions are shown in Table 2. Briefly, 0.5 grams of recrystallized (+)-TBZ or (±)-d6-TBZ, 0.15 grams raw cholesterol (Nippon Fine Chemical, Japan) and 0.35 grams of oil, including sesame, soybean or castor oils (Sigma, US), were added and weighed in vials. All ingredients were mixed rigorously using Kurabo KK-250 planetary mixer-degasser (Kurabo, Japan) with 1600-rpm speed of revolution and rotation for 2 minutes at ambient temperature. The prepared formulations were homogenous, opaque and creamy.

TABLE 2
Composition of VMAT2 inhibitor oleogel-based formulation.

| Formulation | VMAT2 | | VMAT2 | CSTR | Oil |
|---|---|---|---|---|---|
| No. | Inhibitor | Oil | Inhibitor wt % | wt % | wt % |
| F-01 | Rex (+)-TBZ | SSO | 50 | 15 | 35 |
| F-01b | Rex (±)-d6-TBZ | SSO | 50 | 15 | 35 |
| F-01c | Rex (+)-TBZ | SBO | 50 | 15 | 35 |
| F-01d | Rex (+)-TBZ | CTO | 50 | 15 | 35 |

Rex: recrystallized. Rac: racemic. d6: six deuteriums. SSO: Sesame oil. SBO: Soybean oil. CTO: Castor oil. CSTR: cholesterol

### Example 2. VMAT2 Inhibitor Formulations Comprising Coarse Lipid Crystal Structured Oleogels

Preparation and Characterization of 12-Gram Batches of Recrystallized VMAT2 Inhibitors

Dissolved 12 grams of (+)-TBZ in 80 mL ethanol at about 63° C. with moderate mixing in a 120-mL glass vial. Once the solution turned clear and solid particulate was absent, the temperature of the solution was cooled at a speed of ˜2° C./min by coupling a glass double-layer jacket and a water-circulation chiller. The solution was kept agitating at 500 rpm. White precipitates began to be observed at about 42˜45° C. and the suspension was maintained at this temperature for 1 hr. After this isothermal interval, the temperature of VMAT2 inhibitor suspension was decreased at a gradient of 0.2° C./min to 32˜35° C., followed by a temperature gradient of 0.4° C./min to 5° C. Before harvesting, the VMAT2 inhibitor suspension was equilibrated at 5° C. for 2 hours, followed by filtration using a grade 5A 55 mm paper filter (Advantec, Japan) and drying at room temperature. The yield was ˜90%.

The recrystallized (+)-TBZ in the 12-gram batch was observed with microscopy. Briefly, 10 mg/mL (+)-TBZ recrystallized crystal was re-dispersed in 0.2% Tween 20 deionized water with a rigorous vortex. The microscopy was conducted using the optical microscope Leica DMi8, with 100-fold magnification. The PSD was measured using laser diffraction method, with Mastersizer 3000 (Malvern, UK). Before particle sizing, the 10 mg/mL (+)-TBZ crystal was re-dispersed in 0.2% Tween 20 deionized water and sonicated for ˜10 sec.

TABLE 3
Particle size distribution (PSD) of (+)-TBZ recrystallized crystals.

| VMAT2 Inhibitor | D₁₀ (μm) | D₅₀ (μm) | D₉₀ (μm) |
|---|---|---|---|
| 12 g rex (+)-TBZ i | 15.9 | 34.9 | 65.8 |
| 12 g rex (+)-TBZ ii | 17.7 | 40.8 | 80.4 |
| 12 g rex (+)-TBZ iii | 16.7 | 38 | 76 |

Rex: recrystallized. i, ii, and iii: represented three independent preparations

Formulation Composition and Compounding

10 grams of various formulations were prepared in 200 mL plastic cups according to Table 4. Briefly, 4˜7 grams of rex (+)-TBZ, 1.5 to 2.5 grams of cholesterol having a particle size Dv(50) of 27.3 μm and 3˜4 grams of sesame oil or MCT were added and weighed in containers. The compositions were mixed rigorously using Kurabo KK-250 planetary mixer-degasser (Kurabo, Japan) with 1600-rpm speed of revolution and rotation for 2 minutes at ambient temperature.

For preparing formulations containing soybean phosphatidylcholine (SPC) as F-02c and 02d, oleogels were prepared beforehand. SPC was dissolved in either MCT or SSO with the level from 16 to 33%. For enhancing the dissolving rate, the mixing was heated up to 80° C. in an oil bath. Once the SPC-oil solution was transparent, the vehicle was cooled down to room temperature during which SPC was precipitated in SSO and remained transparent in MCT. Vehicles were thus mixed with solid ingredients to prepare formulations. 10 grams of F-02c and 02d were compounded in similar process as F-02, during which 0.5˜3 grams of rex (+)-TBZ, 1 to 2.5 grams of cholesterol and 3˜4 grams of SPC-contained vehicles were added and weighed in containers. The compositions then were mixed rigorously using Kurabo KK-250 planetary mixer-degasser (Kurabo, Japan) with 1600-rpm speed of revolution and rotation for 2 minutes at ambient temperature. The formulations prepared were homogenous, opaque suspensions and some were non-flowable creams or paste. The formulations containing cholesterol showed better physical stability and the homogeneity of the non-flowable creams was maintained much longer. The formulations containing soybean phosphatidylcholine did not show any benefits.

TABLE 4
Composition of VMAT2 inhibitor oleogel-based formulations

| | Rex (+)- | Raw | | | | | |
|---|---|---|---|---|---|---|---|
| Formulation | TBZ | CSTR | SSO | CTO | MCT | SPC | |
| No. | (wt %) | (wt %) | (wt %) | (wt %) | (wt %) | (wt %) | Characteristics |
| F-02 | 50 | 15 | 35 | 0 | 0 | 0 | Non-flowable cream |
| F-02b | 40 | 20 | 40 | 0 | 0 | 0 | Non-flowable cream |
| F-02c | 30 | 10 | 50 | 0 | 0 | 10 | Non-flowable cream |
| F-02d | 15 | 25 | 0 | 0 | 40 | 20 | Gel suspension |
| F-02e | 70 | 0 | 30 | 0 | 0 | 0 | Paste |
| F-02f | 65 | 0 | 0 | 35 | 0 | 0 | Paste |
| F-02g | 65 | 0 | 35 | | 0 | 0 | Non-flowable cream |

SSO: sesame oil,
CTO: Castor oil,
MCT: Medium-chain triglycerides,
SPC: soybean phosphatidylcholine,
CSTR: cholesterol

### Example 3. Jet-Milled VMAT2 Inhibitor Formulations Comprising Coarse Lipid Crystal Structured Oleogels

Preparation and Characterization of Jet Milled (+)-TBZ Crystals

Four grams of raw (+)-TBZ crystals were jet milled in a 20 mm milling chamber at room temperature (Micromacinazione, Switzerland). Briefly, the jet mill was connected to high pressure nitrogen for providing the pneumatic forces. The grinding pressure and feeding pressure are set on 2.5 bar and 5 bar, respectively. The feeding rate of raw (+)-TBZ crystal was 1.6 g/m in. The fine (+)-TBZ powder was collected by the cyclone and recovered in a vial. The yield was ˜80%. The PSD was characterized using laser diffraction method, with Mastersizer 3000 (Malvern, UK). Before particle sizing, the 10 mg/mL (+)-TBZ crystal was re-dispersed in 0.2% Tween 20 deionized water and sonicated for ˜10 sec.

TABLE 5
Particle size distribution (PSD) of
raw and jet milled (+)-TBZ crystals

| (+)-TBZ crystals | D₁₀ (μm) | D₅₀ (μm) | D₉₀ (μm) |
|---|---|---|---|
| Raw (+)-TBZ | 14.3 | 66.5 | 184 |
| Jet milled (+)-TBZ | 2.5 | 5.2 | 11.8 |

Formulation Composition and Compounding

One gram of formulation was prepared in a 20 mL glass vial. The composition is shown in Table 6. Briefly, 0.5 grams of jet milled (+)-TBZ, 0.15 grams of raw cholesterol and 0.35 grams of sesame oil were added and weighed in the vial. All ingredients were mixed rigorously using Kurabo KK-250 planetary mixer-degasser (Kurabo, Japan) with 1600-rpm speed of revolution and rotation for 2 minutes at ambient temperature. The prepared formulation was homogenous, opaque and creamy.

TABLE 6
Composition of jet milled VMAT2 inhibitor
oleogel-based formulation.

| Formulation | Jet milled | Raw | |
|---|---|---|---|
| No. | (+)-TBZ (wt %) | CSTR (wt %) | SSO (wt %) |
| F-03 | 50 | 15 | 35 |

### Example 4. Ball-Milled VMAT2 Inhibitor Formulations Comprising Coarse Lipid Crystal Structured Oleogels

Preparation and Characterization of Ball Milled (±)-TBZ Crystals

5 grams of raw (±)-TBZ crystal (Synnat Pharma, India) was added into a 60 mL vial with 3 cm inner diameter, followed by adding 30 grams of 10 mm Zirconia beads. Then 30 mL of water was added in the vial. The ball milling was operated at 100 rpm and room temperature. The suspension was recovered after about 2 hours of ball milling and lyophilized for recovering the dry (±)-TBZ crystals. The yield of this ball milling was ˜70%. The PSD was characterized using laser diffraction method, with Mastersizer 3000 (Malvern, UK). Before particle sizing, the 10 mg/mL (±)-TBZ crystal was re-dispersed in 0.2% Tween 20 deionized water and sonicated for ˜10 sec.

TABLE 7
Particle size distribution (PSD) of ball milled (±)-TBZ crystals

| (±)-TBZ crystals | D₁₀ (μm) | D₅₀ (μm) | D₉₀ (μm) |
|---|---|---|---|
| Ball milled (±)-TBZ | 4.2 | 9.2 | 20.1 |

Formulation Composition and Compounding

One gram of formulation was prepared in a 20 mL glass vial. The composition is shown in Table 8. Briefly, 0.5 grams of ball milled (±)-TBZ, 0.1 grams of raw cholesterol and 0.4 grams of sesame oil were added and weighed in the vial. All ingredients were mixed rigorously using Kurabo KK-250 planetary mixer-degasser (Kurabo, Japan) with 1600-rpm speed of revolution and rotation for 2 minutes at ambient temperature. The prepared formulation was homogenous, opaque and creamy.

TABLE 8
Composition of ball milled VMAT2 inhibitor
oleogel-based formulation

| Formulation | Ball milled | Raw | |
|---|---|---|---|
| No. | (±)-TBZ (wt %) | CSTR (wt %) | SSO (wt %) |
| F-04 | 50 | 10 | 40 |

### Example 5. VMAT2 Inhibitor Formulations Comprising Jet-Milled Fine Lipid Crystal Structured Oleogels

Preparation and Characterization of Jet-Milled Cholesterol (CSTR)

Four grams of raw cholesterol crystals (Nippon fine chemical, Japan) was jet milled in a 20 mm milling chamber at room temperature (Micromacinazione, Switzerland). The jet mill was connected to high pressure nitrogen for providing the pneumatic forces. The grinding pressure and feeding pressure are set on 2.5 bar and 5 bar, respectively. The feeding rate of raw cholesterol crystal was 1.6 g/m in. The fine CSTR powder was collected by the cyclone and recovered in a vial. The yield was ˜80%. The PSD was characterized using laser diffraction method, with Mastersizer 3000 (Malvern, UK). Before particle sizing, the 10 mg/mL CSTR crystal was re-dispersed in 0.2% Tween 20 deionized water and sonicated for ˜10 sec.

TABLE 10
PSD of raw and jet milled cholesterol crystals

| Cholesterol | D₁₀ (μm) | D₅₀ (μm) | D₉₀ (μm) |
|---|---|---|---|
| Raw CSTR | 14.1 | 27.3 | 48 |
| Jet milled CSTR | 0.3 | 4.6 | 12 |

Formulation Composition and Compounding

One gram of various formulations were prepared in 20 mL glass vials. Compositions are displayed in Table 10. Briefly, 0.5 grams of (+)-TBZ as prepared in example 1, 0.15 or 0.1 grams of jet milled cholesterol and 0.35 or 0.4 grams of sesame oil were added and weighed in vials. All ingredients were mixed rigorously using Kurabo KK-250 planetary mixer-degasser (Kurabo, Japan) with 1600-rpm speed of revolution and rotation for 2 minutes at ambient temperature. The prepared formulations were homogenous, opaque and creamy.

TABLE 10
Composition of VMAT2 inhibitor oleogel-based formulation
containing jet milled cholesterol crystal

| Formulation | Rex | Jet milled | |
|---|---|---|---|
| No. | (+)-TBZ (wt %) | CSTR (wt %) | SSO (wt %) |
| F-05 | 50 | 15 | 35 |
| F-05b | 50 | 10 | 40 |

Rex: recrystallized

### Example 6. VMAT2 Inhibitor Formulations Comprising Spray-Dried Fine Lipid Crystal Structured Oleogels

Preparation and Characterization of Spray Dried Cholesterol

The spray drying was conducted with Mini Spray Dryer B-290, (Buchi, Switzerland). First, 10 grams of raw cholesterol crystal was dissolved in 300 mL isopropanol (IPA) with 35-40° C. heating to obtain a clear solution. Operation parameters of spray drying are listed in Table 11. The spray drying was conducted at a flow rate of 12.5 ml/min and completed in 2 hours. The fine CSTR particles were collected by the cyclone and recovered in a vial. The yield was about 80%. The spray dried CSTR (SPD CSTR) was observed with microscopy. Briefly, 10 mg/mL SPD CSTR was re-dispersed in 0.2% Tween 20 deionized water with a rigorous vortex. The microscopy was conducted using the optical microscope Leica DMi8, with 100-fold magnification. The PSD was characterized using laser diffraction method, with Mastersizer 3000 (Malvern, UK) and the results are shown in Table 12. Before particle sizing, the 10 mg/mL SPD CSTR was re-dispersed in 0.2% Tween 20 deionized water and sonicated for ˜10 sec.

TABLE 11
Parameters of preparing spray dried cholesterol

| Parameters | Dissolving solvent | Inlet/Outlet temp (° C.) |
|---|---|---|
| Experimental factor | IPA | 100/80 |

TABLE 12
PSD of spray dried cholesterol

| Cholesterol | D₁₀ (μm) | D₅₀ (μm) | D₉₀ (μm) |
|---|---|---|---|
| Spray dried CSTR | 0.2 | 2.0 | 5.0 |

Formulation Composition and Compounding

One gram of various formulations were prepared in 20 mL glass vials. Compositions are listed in Table 13. Briefly, 0.5 grams of recrystallized (+)-TBZ as described in example 1, 0.15 or 0.1 gram of spray dried cholesterol and 0.35 or 0.4 grams of sesame oil were added and weighed in vials. All ingredients were mixed rigorously using Kurabo KK-250 planetary mixer-degasser (Kurabo, Japan) with 1600-rpm speed of revolution and rotation for 2 minutes at ambient temperature. The prepared formulations were homogenous, opaque and creamy.

TABLE 13
Composition of VMAT2 inhibitor oleogel-based formulation
containing spray dried cholesterol crystal

| Formulation No. | Rex (+)-TBZ wt % | SPD CSTR wt % | SSO wt % |
|---|---|---|---|
| F-06 | 50 | 15 | 35 |
| F-06b | 50 | 10 | 40 |

### Example 7. Recrystallized VMAT2 Inhibitor Formulations Comprising High Shear Re-Dispersed Lipid Fiber Structured Oleogels

Preparation and Characterization of Cholesterol Fiber Structured Oleogel

2 grams of cholesterol fiber (FIG. 4) oleogel (CFROG) was prepared by dispersing solid cholesterol fiber (CF) in oil (including SSO, MCT and CTO) using IKA T10 rotor stator homogenizer at the speed of 30,000 rpm for 20 minutes, against water bath at room temperature. The composition of CF dispersion oleogel is listed in Table 14. The resulting oleogel is white, non-flowable but injectable.

TABLE 14
Compositions of cholesterol fiber re-dispersed oleogel

| CFROG No. | Oil | CSTR fiber solid wt % | Oil wt % |
|---|---|---|---|
| CFROG-01 | SSO | 10 | 90 |
| CFROG-02 | SSO | 20 | 80 |
| CFROG-03 | SSO | 30 | 70 |
| CFROG-04 | MCT | 20 | 80 |
| CFROG-05 | CTO | 10 | 90 |

CFROG: cholesterol fiber re-dispersed oleogel. SSO: sesame oil, CTO: Castor oil, MCT: Medium-chain triglycerides, CSTR: cholesterol

Formulation Composition and Compounding

One gram of various formulations were prepared in 20 mL glass vials. Compositions are shown in Table 15. 0.5 grams of recrystallized (+)-TBZ or (±)-d6 TBZ and 0.5 grams of cholesterol-based oleogels were added and weighed. All ingredients were mixed using Kurabo KK-250 planetary mixer-degasser (Kurabo, Japan) with 1600-rpm speed of revolution and rotation for 2 minutes at ambient temperature. The prepared formulation was homogenous and opaque.

TABLE 15
Compositions of VMAT2 inhibitor formulations

| Formu- | | | | CSTR |
|---|---|---|---|---|
| lation | | CFROG | | fiber |
| No. | VMAT2 Inhibitor/wt % | No. | Oil/wt % | wt % |
| F-07 | Rex (+)-TBZ/50 | CFROG-02 | SSO/40 | 10 |
| F-07b | Rex (+)-TBZ/50 | CFROG-03 | SSO/35 | 15 |
| F-07c | Rex (+)-TBZ/50 | CFROG-04 | MCT/40 | 10 |
| F-07d | Rex (±)-d6-TBZ/50 | CFROG-03 | SSO/35 | 15 |

### Example 8. VMAT2 Inhibitor Formulations Comprising Lipid Fiber Structured Oleogels

Formulation Composition and Compounding

2 grams of (+)-TBZ crystals, 0.4 grams of dried, cholesterol fiber-shaped (CF) particles, and 1.6 grams of sesame oil (Sigma, US) were added and weighed in a 20 ml vial. All ingredients were mixed rigorously using Kurabo KK-250 planetary mixer-degasser (Kurabo, Japan) with 1600-rpm speed of revolution and rotation for 2 minutes at ambient temperature. The prepared formulations were homogenous, opaque and creamy. The compositions of VMAT2 inhibitor formulations are described in Table 16.

TABLE 16
Compositions of VMAT2 inhibitor formulation

| Formulation No. | (+)-TBZ wt % | CSTR fiber wt % | SSO wt % |
|---|---|---|---|
| F-08 | 50 | 10 | 40 |

Characterization of Recrystallized Crystals in the Formulation

The formulation was characterized by both microscopy and laser diffraction. The microscopic sample was prepared by spreading F-08 on glass slides using a spatula without additional treatment or dilution. There is no cover slip on the sample. The microscopy was conducted using the optical microscope Leica DMi8, with 100-fold magnification.

The PSD was measured using laser diffraction method, with Mastersizer 3000 (Malvern, UK). The particle sizing samples were prepared by re-dispersing 25 mg of F-08 in 1 mL (+)-TBZ-saturated heptane. The purpose of (+)-TBZ-saturated heptane is to dissolve structured cholesterol fiber and oil without affecting (+)-TBZ particles. The principle is based on the difference of solubility of cholesterol and (+)-TBZ in heptane. The solubility of cholesterol and (+)-TBZ in heptane at room temperature are >10% and <0.4% by weight, respectively. The (+)-TBZ saturation is further used to prevent the dissolving (+)-TBZ during the particle sizing. The clear (+)-TBZ-saturated heptane solution was prepared by filtering 10 mg/mL (+)-TBZ suspension using 0.2 μm filter. The suspension was rigorously mixed for 1 hour before filtering. During the laser diffraction measurement, the 7-mL cuvette was filled with (+)-TBZ-saturated heptane. The PSD results of (+)-TBZ crystals in F-08 are displayed in Table 17.

TABLE 17
PSD of (+)-TBZ particles in sample F-08

| Formulation | D₁₀ (μm) | D₅₀ (μm) | D₉₀ (μm) |
|---|---|---|---|
| F-08-i | 15.8 | 29.9 | 51.8 |
| F-08-ii | 15.2 | 27.4 | 46.5 |
| F-08-iii | 13.9 | 25.8 | 45.2 |

Note:
i, ii and iii represent three independent batches.

Viscosity Evaluation of the Formulation

The viscosity of F-08 was measured by Anton Paar Rheometer MCR302e (Anton Paar, Austria) at 25° C. The measured spindle is PP20. About 1 gram of F-08 was loaded on the temperature control plate and then contact to the spindle. The viscosity was measured with increasing rotational speed, which is proportional to increasing shear rate. The viscosity-shear rate profile was plotted accordingly and shown in FIG. 6. The shear thinning factor (maximum viscosity at low shear and minimum viscosity at high shear) was as high as 100-fold.

### Example 9. Injection Force Evaluation of VMAT2 Inhibitor Cholesterol Structured Oleogel Formulations

The preparation methods of measured formulation, F-06, F-07 and F-07b have been described in examples 6 and 7. The test article was filled into standard 1 mL long syringes (complied to ISO 11040-6 such as those supplied by Schott or Terumo), and then coupled to needles (HenkeSassWolf, Germany) via the leur lock screw and sealed by West 4023/50 plungers. The sizes of tested needles include 18 gauge regular-walled and 21 Gauge thin-walled needles. During injection force measurement, the syringe is mounted on a fixture at room temperature and the force was evaluated using a Yotec FSH-500N force gauge (max tolerated force is 500N). The push speed applied to the plunger was set at 4.5 cm/min. The results of injection force and equipped needles are described in Table 18.

TABLE 18
Injection force of VMAT2 inhibitor formulations.

| Formulation | | | Gliding |
|---|---|---|---|
| ID | Formulation Composition | Needle | force (N) |
| F-06 | (50 wt % rex (+)-TBZ/15 wt % | 18G-RW × | 26 |
| | SPD CSTR/35 wt % SSO) | 16 mm | |
| F-07 | (50 wt % rex (+)-TBZ/10 wt % | 18G-RW × | 18 |
| | CSTR fiber/40 wt % SSO) | 16 mm | |
| F-07 | (50 wt % rex (+)-TBZ/10 wt % | 21G-TW × | 24 |
| | CSTR fiber/40 wt % SSO) | 16 mm | |
| F-07b | (50 wt % rex (+)-TBZ/15 wt % | 21G-TW × | 35 |
| | CSTR fiber/35 wt % SSO) | 16 mm | |

### Example 10. Stability of VMAT2 Inhibitor Cholesterol Structured Oleogel Formulation

The preparation methods of measured formulation, F-06 and F-07b have been described in examples 6 and 7. For providing sufficient studied syringes, 5 grams of formulation were prepared, instead of the 1-gram process. About 400 mg of formulations F-06 and F-07b were each filled into 1 mL Long plastic syringe (Schott, Switzerland) and sealed by West 4023/50 plungers and Sumitomo P101A tip cap. Before transferring to storage environment at varied temperatures, the filled F-06 and F-07b were sterilized by 30 kGy gamma radiation. The purity was evaluated using UPLC and quantitatively defined as the area fraction of the intact TBZ peak to total peak areas. The detailed parameters of the method are displayed in Table 19. The results of purities are shown in Table 20.

TABLE 19
Parameters LC and the gradient of eluting mobile phase.

| Parameters | Description | |
|---|---|---|
| LC instrument | Shimadzu LC-30AD with PDA | |
| Column | Waters Acquity UPLC BEH C18 (150 × 3 mm) | |
| Mobile phase A (MPA) | 0.5 mM ammonium acetate | |
| | (Aq.):Acetonitrile = 7:3 | |
| Mobile phase B (MBP) | IPA | |
| Flow rate | 0.3 | mL/min |
| Column Temp. | 55° | C. |
| Injection Volume | 2 | uL |
| Detection wavelength | 220 | nm |
| Run Time | 27 | min |

Elution Gradient

| Time (min) | % of MPB |
|---|---|
| 0 | 5 |
| 5 | 30 |
| 15 | 60 |
| 16 | 95 |
| 20 | 100 |
| 22 | 5 |
| 27 | 5 |

TABLE 20
Purity of VMAT2 inhibitor formulations, F-06 and
F-07b under storage conditions and durations

| Formulation | Stress (Temp./Time) | Purity (%) |
|---|---|---|
| F-06 | Post 30KGγ gamma before stress | 99.47 |
| (50 wt % rex (+)-TBZ/ | 25° C./1 month | 99.34 |
| 15 wt % SPD CSTR/ | 25° C./3 month | 99.26 |
| 35 wt % SSO) | 40° C./1 month | 99.35 |
| | 40° C./3 month | 99.07 |
| | 60° C./1 month | 99.18 |
| | 60° C./2 month | 98.52 |
| F-07b | Post 30KGγ gamma before stress | 99.38 |
| (50 wt % rex (+)-TBZ/ | 25° C./2 month | 99.25 |
| 15 wt % CSTR fiber/ | 25° C./3 month | 99.1 |
| 35 wt % SSO) | 40° C./1 month | 99.02 |
| | 40° C./2 month | 98.87 |
| | 40° C./3 month | 98.74 |
| | 60° C./1 wk | 99.05 |
| | 60° C./1 month | 98.05 |

### Example 11. In Vitro Dissolution of VMAT2 Inhibitor Cholesterol Structured Oleogel Formulations

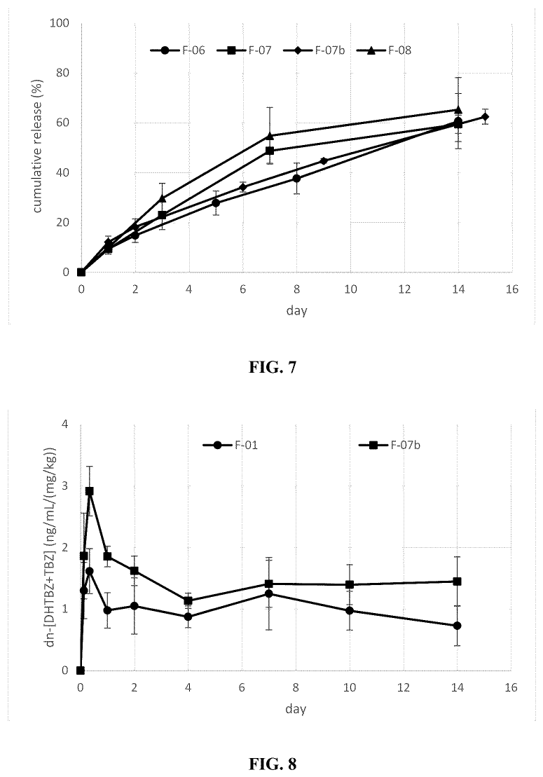

The preparation methods of studied formulation, F-06, F-07, F-07b and F-08 have been described in examples 6, 7 and 8. About 10 to 100 mg of test articles were dosed in the dissolution medium, comprising 1× phosphate buffered saline (PBS) supplemented with 0.1% sodium azide and 0.2% Tween 20. The dissolution was done in 400 ml PBS medium at pH 7.4 in 500 ml vials. These dissolution vials were then incubated at 37° C. and agitated by moderate rotation at 100 rpm. The dissolution media were sampled at different time points and assayed using UPLC to evaluate the released VMAT2 inhibitors. The parameters in assay method are displayed in Table 21. The cumulative release profiles are plotted in FIG. 7. The sustained release of (+)-TBZ in vitro more than two weeks was observed.

TABLE 21
LC parameters and the gradient of elution in the assay method

| Parameters | Description | |
|---|---|---|
| LC instrument | Shimadzu LC-30AD with PDA | |
| Column | Waters Acquity UPLC BEH C18 (150 × 3 mm) | |
| Mobile phase A (MPA) | 0.5 mM ammonium acetate | |
| | (Aq.):Acetonitrile = 1:1 | |
| Mobile phase B (MBP) | IPA | |
| Flow rate | 0.3 | mL/min |
| Column Temp. | 55° | C. |
| Injection Volume | 2 | uL |
| Detection wavelength | 220 | nm |
| Run Time | 10 | min |

Elution Gradient

| Time (min) | % of MPB |
|---|---|
| 0 | 35 |
| 4 | 90 |
| 6 | 95 |
| 6.5 | 35 |
| 10 | 35 |

### Example 12. 2-Week PK of VMAT2 Inhibitor Cholesterol Structured Oleogel Formulations in Rats

The preparation methods of studied formulation, F-01 (50 wt % rex (+)-TBZ/15 wt % raw CSTR/35 wt % SSO) and F-07b (50 wt % rex (+)-TBZ/15 wt % CSTR fiber/35 wt % SSO) have been described in examples 1 and 7. For providing sufficient studied syringes, 2 grams of formulation were prepared, instead of the 1-gram process. The test formulations for the PK profile study were sterilized by 30 kGy gamma radiation before dosing. The studied animals were male Sprague-Dawley rats, with >300 grams body weight. For each tested formulation, four rats were administrated subcutaneously at the level of ˜60 mg/kg through the site of dorsal thoracic. Blood samples were collected by bleeding the lateral tail veins with EDTA disodium as anti-coagulant. Collected blood samples were centrifuged for 15 min at 1000×g within 60 minutes after blood collection. The plasma samples were transferred to labeled tubes and stored in a freezer at temperature below −60° C. Plasma level of TBZ and DHTBZ in samples was measured using LC-MS/MS. PK profiles are shown in FIG. 8. The sustained release of TBZ and DHTBZ in vivo lasted longer than two weeks.

### Example 13. 5-Week Pharmacokinetic (PK) Study of VMAT2 Inhibitor Cholesterol Structured Oleogel Formulations in Rats

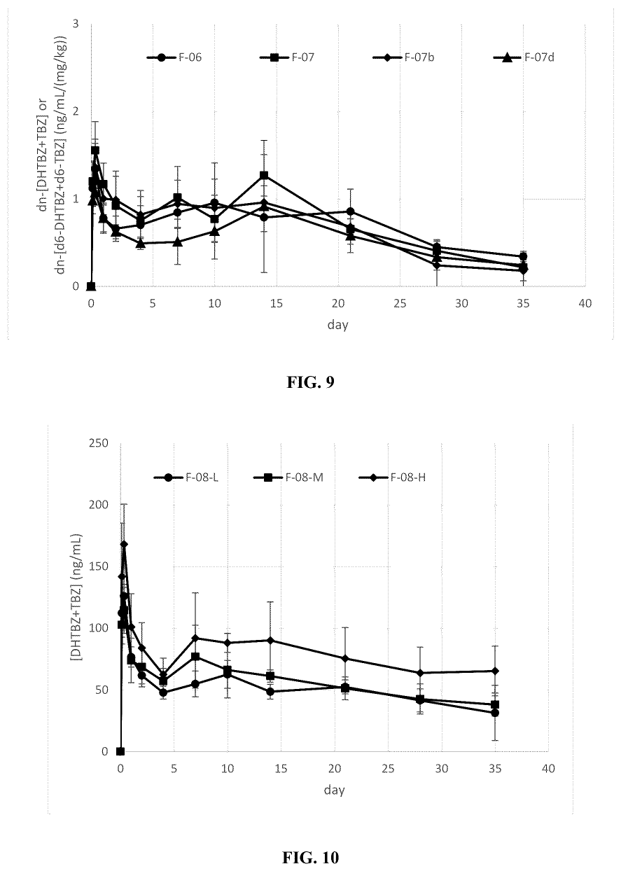

The in vivo method of 5-week studies can be referred to PK studies described in example 12, except for the 5 weeks of PK profile evaluation and the LC evaluation of (±)-d6-TBZ and (±)-d6-DHTBZ. The preparation methods of studied formulation, F-06, F-07, F-07b and F-07d have been described in examples 6 and 7. For providing sufficient studied syringes, 2 grams of formulation were prepared, instead of the 1-gram process. The test formulations for the PK profile study were sterilized by 30 kGy gamma radiation before dosing. PK profiles are plotted as FIG. 9. The sustained release of TBZ and DHTBZ or deuterated in vivo longer than five weeks was demonstrated.

### Example 14. Dose-Proportionality PK of VMAT2 Inhibitor Cholesterol Structured Oleogel Formulations in Rats

The in vivo method for dose proportionality studies can be referred to PK studies described in example 12, except for the dose levels ranged from 70 to 130 mg/kg and the dose formulation is F-08. The preparation methods of studied formulation, F-08 has been described in examples 8. The test formulations for the PK profile study were sterilized by 30 kGy gamma radiation before dosing. The PK profiles at different doses were displayed in FIG. 10. The results of AUC dose proportionality are shown in Table 22.

TABLE 22
AUC of VMAT2 inhibitor formulation, F-08.

| Dose (mg/kg) | AUC_{35 d} (ng/mL) × day |
|---|---|
| 76 | 1765 |
| 106 | 1963 |
| 133 | 2745 |

### Example 15. PK Profiles of VMAT2 Inhibitor Cholesterol Structured Oleogel Formulations in Dogs

The preparation method of studied formulation, F-06 has been described in example 6. For providing sufficient studied syringes, 2 grams of formulation were prepared, instead of the 1-gram process. The test formulation for the PK profile study were sterilized by 30 kGy gamma radiation before dosing. The PK study was conducted in male beagle dog, with body weight >10 kg. For each tested formulation, three beagles were administrated subcutaneously at the level of ˜10 mg/kg through the site of dorsal thoracic. Blood samples were collected by bleeding the cephalic veins with EDTA disodium as anti-coagulant. Collected blood samples was centrifuged for 15 min at 1000×g within 60 minutes after blood collection. The plasma samples were transferred to labeled tubes and stored in a freezer at temperature below −60° C. Plasma levels of (+)-TBZ and (+)-α-DHTBZ, in samples were measured using LC-MS/MS. PK profiles are plotted in FIG. 11. It seems that the release of (+)-TBZ and (+)-α-DHTBZ can last much longer in dogs than in rats.

### Example 16. Preparation of (+)-α-DHTBZ-Based Oleogel Formulations

4 grams of raw (+)-α-DHTBZ crystals (Wuxi Pharma, China) were jet milled in a 20 mm milling chamber at room temperature (Micromacinazione, Switzerland). Briefly, the jet mill was connected to high pressure nitrogen for providing pneumatic forces, with grinding pressure and feeding pressure set at 4 and 5 bars, respectively. The feeding rate of raw (+)-α-DHTBZ crystals was 1.6 g/m in. Fine milled (+)-α-DHTBZ crystals were collected by the cyclone and recovered in a vial. The yield was ˜80%. The PSD of jet-milled (+)-α-DHTBZ was characterized using laser diffraction method, with Mastersizer 3000 (Malvern, UK). Before particle sizing, the 10 mg jet milled (+)-α-DHTBZ was re-dispersed in 1 mL of 0.2% Tween-20 deionized water and sonicated for ˜10 sec. The PSD of jet milled α-DHTBZ is shown in Table 23.

TABLE 23
PSD of jet milled (+)-α-DHTBZ

| VMAT2 Inhibitor | D₁₀ (μm) | D₅₀ (μm) | D₉₀ (μm) |
|---|---|---|---|
| jet milled α-DHTBZ | 5 | 13 | 40 |

Oleogel vehicle was prepared before addition of active drug substance. Oleogel vehicles containing SPC in a range from 30% to 60% in oils were prepared by dissolving SPC in oils including MCT and SSO. For enhancing the dissolving rate, SPC/oil mixing was heated up to 80° C. using an oil bath. Once SPC/oil solutions became transparent, vehicles were cooled down to room temperature during which SPC was precipitated in SSO and remained transparent in MCT. Vehicles were then mixed with solid active ingredients and optionally lipid to prepare formulations.

1 gram of F-DH-01 and F-DH-02 were prepared in 20 mL glass vials. Briefly, 0.5 grams of jet milled (+)-α-DHTBZ and 0.5 grams of MCT/SPC and SSO/SPC vehicles were added and weighed in vials. The compositions were mixed rigorously using Kurabo KK-250 planetary mixer-degasser (Kurabo, Japan) with 1600-rpm speed of revolution and rotation for 2 minutes at ambient temperature. 1 gram of F-DH-03 was prepared in similar process as F-DH-01, except that the 0.5 grams solid comprises 0.35 grams of jet milled α-DHTBZ and 0.15 grams of cholesterol.

TABLE 24
Injectable (+)-α-DHTBZ oleogel-based formulations.

| | (+)-α- | | | | | |
|---|---|---|---|---|---|---|
| Formulation | DHTBZ | MCT | SSO | SPC | CSTR | |
| No. | (wt %) | (wt %) | (wt %) | (wt %) | (wt %) | Characteristics |
| F-DH-01 | 50 | 20 | 0 | 30 | 0 | Non-flowable cream |
| F-DH-02 | 40 | 0 | 42 | 18 | 0 | Non-flowable cream |
| F-DH-03 | 35 | 25 | 0 | 25 | 15 | Paste |

SSO: sesame oil,
SPC: soybean phosphatidylcholine,
CSTR: cholesterol

### Example 17. PK Profile of Sustained-Release (+)-α-DHTBZ-Based Oleogel Formulations

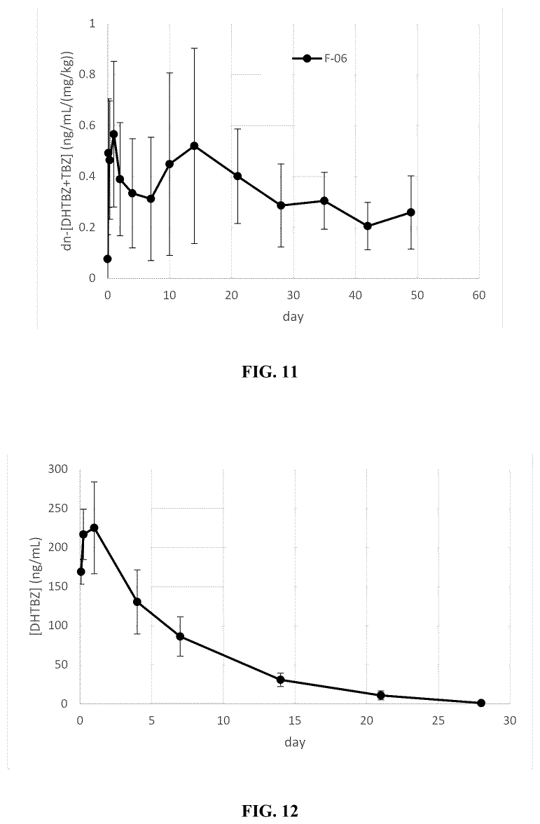

2 grams of F-DH-01 was prepared as process carried out in 1 g formulation preparation, except for the 2-fold quantity of materials. F-DH-01 for the PK profile study was sterilized by 30 kGy gamma radiation before dosing. The studied animals were male Sprague-Dawley rats, with >300 grams body weight. Four rats were administrated subcutaneously at the level of ˜60 mg/kg through the site of dorsal thoracic. Blood samples were collected by bleeding the lateral tail veins with EDTA disodium as anti-coagulant. Collected blood samples were centrifuged for 15 min at 1000×g within 60 minutes after blood collection. The plasma samples were transferred to labeled tubes and stored in a freezer at temperature below −60° C. Plasma level of (+)-α-DHTBZ in samples was measured using LC-MS/MS. PK profiles are shown in FIG. 12. The release duration of (+)-α-DHTBZ can last for about one month.

What is claimed:

1. A depot composition comprising: a) a vesicular monoamine transporter 2 (VMAT2) inhibitor or a pharmaceutically acceptable salt thereof; b) a pharmaceutically acceptable oil; and c) a lipid, wherein the lipid is a solid at a temperature between about 15 and about 30° C., and the lipid is present in an amount from 0% to about 50% by weight, relative to the weight of the depot composition.

2. The depot composition of claim 1, wherein the VMAT2 inhibitor is selected from a group consisting of VBZ, (±)-TBZ, (+)-TBZ, (−)-TBZ, (±)-d6-TBZ, (+)-d6-TBZ and (−)-d6-TBZ.

3. The depot composition of claim 1, wherein the VMAT2 inhibitor is selected from a group consisting of (+)-(α)-DHTBZ, (−)-(α)-DHTBZ, (+)-(β)-DHTBZ, (−)-(β)-DHTBZ, (+)-d6-(α)-DHTBZ, (−)-d6-(α)-DHTBZ, (+)-d6-(β)-DHTBZ, and (−)-d6-(β)-DHTBZ.

4. The depot composition of claim 1, wherein the VMAT2 inhibitor is (+)-TBZ.

5. The depot composition of claim 1, wherein the pharmaceutically acceptable oil is selected from the group consisting of triglycerides, vegetable oils, sesame oil, soybean oil, cottonseed oil, castor oil, olive oil, medium chain triglyceride (MCT), dioleate glycerol, ethyl oleate and ethyl linoleate.

6. The depot composition of claim 1, wherein the lipid is selected from the group consisting of sterols, natural waxes, synthetic waxes, hydroxysteroid, and ketosteroid.

7. The depot composition of claim 1, wherein the lipid is cholesterol.

8. The depot composition of claim 1, wherein the VMAT2 inhibitor or the pharmaceutically acceptable salt thereof is present in an amount of less than about 70% by weight, relative to the weight of the depot composition.

9. The depot composition of claim 1, wherein the ratio of the lipid to the pharmaceutically acceptable oil is between about 1:1 and about 1:99 by weight.

10. The depot composition of claim 1, wherein the lipid has a particle size distribution characterized by D(50) in a range from about 1 μm to about 100 μm.

11. The depot composition of claim 1, wherein the VMAT2 inhibitor or the pharmaceutically acceptable salt thereof has a particle size distribution characterized by D(50) in a range from about 3 μm to about 300 μm.

12. The depot composition of claim 1, wherein the VAMT2 inhibitor or the pharmaceutically acceptable salt thereof is dispersed in the depot composition that is filled in a syringe for subcutaneous or intramuscular injection.

13. A method of treating a hyperkinetic movement disorder comprising administering to a patient in need thereof the depot composition of claim 1 via injection.

14. The method of claim 13, wherein an in situ sustained release depot is formed upon the administering to the patient, and the VMAT2 inhibitor or the pharmaceutically acceptable salt thereof is released to the patient for at least one week.

15. The method of claim 14, which, after the administering, provides the release of the VMAT2 inhibitor or the pharmaceutically acceptable salt thereof with a plasma level peak/trough (P/T) ratio between about 1 to about 20.

16. The method of claim 14, wherein the release of the VMAT2 inhibitor or the pharmaceutically acceptable salt thereof from the in situ sustained release depot is no more than about 30% of total amount of the VMAT2 inhibitor or the pharmaceutically acceptable salt thereof within 24 hours after the administration.

17. The method of claim 13, wherein the hyperkinetic movement disorder is selected from the group consisting of tardive dyskinesia (TD), chorea associated with Huntington's disease (HD), tremors, dystonia, tics, myoclonus, stereotypies, restless legs syndrome, and various other disorders with abnormal involuntary movements.

18. The depot composition of claim 1, wherein the depot composition has a viscosity between about 300,000 and about 10,000,000 mPas at room temperature when measured at a shear <0.1 s⁻¹ and the viscosity is between about 1,000 and about 50,000 mPas at room temperature when measured at a shear >10 s⁻¹.
